(19) United States
(12) Patent Application Publication
(10) Pub. No.: US 2008/0269310 A1
(43) Pub. Date: Oct. 30, 2008

(54) Synergistic Combinations

(76) Inventor: Adrian Paul Foster, Kent (GB)

Correspondence Address: PFIZER INC., PATENT DEPARTMENT, MS8260-1611, EASTERN POINT ROAD, GROTON, CT, 06340, US

(21) Appl. No.: 11/914,825

(22) PCT Filed: May 18, 2006
(86) PCT No.: PCT/IB2006/001655

§ 371 Date: Jun. 27, 2008

Related U.S. Application Data
(60) Provisional application No. 60/683,391, filed on May 20, 2005.

(51) Int. Cl. A61K 31/403; A61K 31/195; A61P 39/00; A61K 31/197

(52) U.S. Cl. 514/411; 514/561

(57) ABSTRACT

This invention relates to synergistic combinations of non-steroidal anti [symbol] inflammatory drugs, particularly carprofen, with alpha-2-delta ligands, such as gabapentin or pregabalin, for veterinary treatment of pain and/or inflammation, particularly in dogs, cats and horses.

### FIELD OF THE INVENTION

This invention relates to combinations of non-steroidal anti-inflammatory drugs (NSAIDs) and alpha-2-delta ligands, particularly carprofen and an alpha-2-delta ligand, such as gabapentin or pregabalin. More particularly, the invention relates to combinations of a NSAID and alpha-2-delta ligand which exhibit a synergistic effect and the use of such combinations for veterinary treatment of pain and/or inflammation in mammals, particularly in dogs, cats and horses.

### BACKGROUND TO THE INVENTION

The use of NSAIDs in dogs, cats and horses has been more limited in comparison to the use of NSAIDs in humans. Consequently, there is less experience and knowledge in veterinary medicine about safety and efficacy issues surrounding the use of NSAIDs in these animals. In veterinary medicine, for example, the most common indications for NSAIDs are degenerative joint disease (DJD) and osteoarthritis, which, in dogs, cats and horses, often results from a variety of development diseases as well as from traumatic injuries to joints. In addition to the treatment of chronic pain and inflammation, NSAIDs are also useful for treating post-surgical acute pain in dogs, cats and horses.

NSAIDs have been approved in a number of countries for use in dogs, (Carprofen, Ketoprofen, Meloxicam, Phenylbutazone, Etodolac); cats (Carprofen, Ketoprofen, Dipyrone) and horses (Carprofen, Ketoprofen, Flunixin). However, there are reports that depending on the nature and severity of the disease patients are, or become, refractory to NSAIDs. Carprofen (6-chloro-α-methyl-9H-carbazole-2-acetic acid) is a NSAID approved for use in veterinary medicine. It is disclosed in U.S. Pat. No. 3,896,145, and is widely marketed as Rimadyl®. Carprofen has the following structural formula:

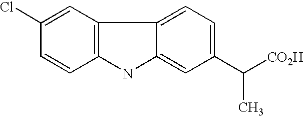

Carprofen is known to inhibit COX-2 in dogs, according to in vitro, ex vivo and in vivo measurement (WO 98/50033, AJVR, 1998, 59, 11, 1441), although other reports suggest alternative mechanisms of action. It is used in dogs for the treatment of analgesia, inflammation and fever, particularly for the relief of pain and inflammation associated with osteoarthritis and for the control of postoperative pain associated with soft tissue and orthopaedic surgeries. It has been used widely for the treatment of dogs and humans, (Irish Veterinary Journal, 1999, 52, 11, 606, Veterinary Record, 2002, 150, 684, JAVMA, 1997, 210, 10, 1493, Vet Comp Orthop Traumatol, 1997, 10, 122). Carprofen is authorised in some countries for use in the cat for the control of postoperative pain. It has also been studied in the horse, and is approved for use in horses for treatment of musculoskeletal pain and post-operative surgery.

It is known that carprofen, especially the (S)-enantiomer of carprofen, and related compounds have a surprising and unexpectedly high degree of selectivity for the COX-2 isozyme compared to the COX-1 isozyme in dogs WO 98/50033, (129 fold selectivity), American Journal Veterinary Research, 2002, 63, 1, 91 (16.8 fold selectivity), American Journal of Veterinary Research, 2001, 62, 11, 1755, (6.5 fold selective), American Journal Veterinary Research, 2000, 61, 7, 802 (1.75 fold selective). Selectivity for the COX-2 isozyme compared to the COX-1 isozyme in cats has been reported to be 5.5 fold and in horses 1.6 fold, American Journal of Veterinary Research, 2001, 62, 11, 1755. While the COX-2 isozyme is an important mediator of inflammation, there are many other important mediators of inflammation that either have no interaction with NSAIDs, or no well understood relationship to the action of NSAIDs. Such mediators include several classes of leukocytes; cell adhesion molecules; soluble mediators such as C5a, PAF and leukotriene B4; cytokines such as IL-1 and TNF; growth factors such as GMCSF and TGFbeta; histamine, bradykinin and 5HT. While the compounds of formula (III) have been shown to be unique inhibitors of COX-2, there is no intention thereby to be bound to any particular mechanism of action by which the compounds of formula (III) might exert their anti-inflammatory activity.

Alpha-2-delta ligands may be defined as compounds, which selectively displace ³H-gabapentin from porcine brain membranes, thereby indicating a high affinity interaction with the alpha-2-delta (α₂δ) subunit of voltage-gated calcium channels. Alpha-2-delta ligands also include compounds which do not displace ³H-gabapentin, but which are structurally similar to compounds that do, and which might be expected to bind to the alpha-2-delta subunit at a different site than ³H-gabapentin, or may bind to human brain alpha-2-delta or that of companion animals, such as dogs, cats and horses, but not to porcine alpha-2-delta. Such compounds may also be known as GABA analogs.

Alpha-2-delta ligands have been described for a number of indications in humans, including epilepsy, neuropathic pain, anxiety, and fibromyalgia. The best known alpha-2-delta ligand, gabapentin (Neurontin®), 1-(aminomethyl)-cyclohexylacetic acid, was first described in the patent literature in the patent family comprising U.S. Pat. No. 4,024,175. Gabapentin has been studied for the treatment of pain (British Journal of Pharmacology, 2000, 131, 2, 282, Anaesthesia, 2002, 57, 451, Brain Research, 1998, 810, 93, British Journal of Pharmacology, 1997, 121, 1513, Clin J Pain, 2001, 17, 4, 284, Rev Neurol (Paris), 1997, 153, 1S, 39), and is approved for the treatment of epilepsy and neuropathic pain.

US 2002068718 discloses oral compositions containing hyaluronic acid or its salts, optionally containing a therapeutic drug such as gabapentin for the treatment or prevention of osteoarthritis, joint effusion, joint inflammation, and pain. The compositions are intended to be chondroprotective or restorative.

A second alpha-2-delta ligand, pregabalin, (S)-(+)-4-amino-3-(2-methylpropyl)butanoic acid, (Lyrica®) is described in European patent application publication number EP641330 as an anti-convulsant treatment useful in the treatment of epilepsy. Pregabalin also is described in EP0934061 for the treatment of pain. Pregabalin also has been described for use in treating anxiety in humans.

No alpha-2-delta ligands have been approved for use in veterinary species. Some work has been undertaken to investigate the antiepileptic effect of gabapentin on small animals (Clinical techniques in small animal practice, 1998, 13, 3, 185; Veterinary Clinics of North America. Small animal practice, 1998, 28, 2, 411, Veterinary Clinics of North America. Small animal practice, 2000, 30, 1, 183).

The effect of a combination of an alpha-2-delta ligand, gabapentin, and a NSAID, ibuprofen, on pain has been investigated and the effect was additive when these agents were delivered concurrently (Anaesthesiology, 1999, 91, 1006). Co-administration of naproxen and gabapentin or naproxen and pregabalin in a rodent model of hyperalgesia has been described in (WO 99/12537, WO 2000053225, Anaesthesiology, 2002, 97, 5, 1263).

Work has been carried out on co-administration of NSAIDs with antiepileptics to protect against convulsive activity, Pharmacological Research, 1998, 37, 5, 375, Polish Journal of Pharmacology, 1998, 50, 1, 94.

There remains an unmet need for an improved, yet safe, treatment of pain and/or inflammation in cats, dogs and horses.

### SUMMARY OF THE INVENTION

According to one aspect the present invention provides the use of an alpha-2-delta ligand of formula (I)

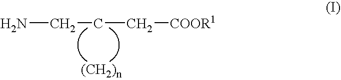

wherein R¹ is hydrogen or (C₁-C₄)alkyl; n is an integer of from 4 to 6; or a pharmaceutically acceptable salt or solvate thereof, or an alpha-2-delta ligand of formula (II)

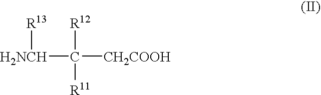

wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; in combination with a non-steroidal anti-inflammatory compound of formula (III)

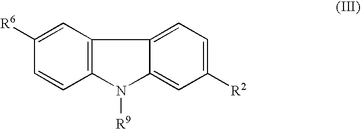

wherein R² is

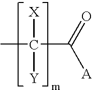

wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof, in the manufacture of a medicament for the curative, prophylactic or palliative treatment of pain and/or inflammation in a mammal selected from cats, dogs and horses.

According to another aspect the invention provides a method for the curative, prophylactic or palliative treatment of pain and/or inflammation in a mammal selected from cats, dogs and horses comprising administering to the mammal an effective amount of an alpha-2-delta ligand of formula (I)

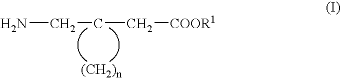

wherein R¹ is hydrogen or (C₁-C₄)alkyl; n is an integer of from 4 to 6; or a pharmaceutically acceptable salt or solvate thereof, or an alpha-2-delta ligand of formula (II)

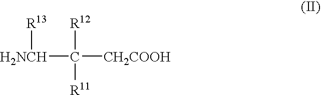

wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; in combination with a non-steroidal anti-inflammatory compound of formula (III)

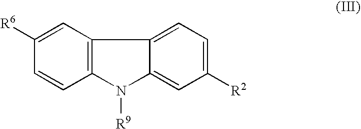

wherein R² is

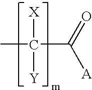

wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof.

According to another aspect the invention provides a pharmaceutical composition comprising an alpha-2-delta ligand of formula (I)

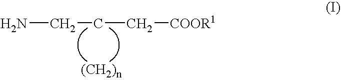

wherein R¹ is hydrogen or (C₁-C₄)alkyl; n is an integer of from 4 to 6; or a pharmaceutically acceptable salt or solvate thereof, or an alpha-2-delta ligand of formula (II)

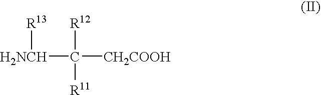

wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; and a non-steroidal anti-inflammatory compound of formula (III)

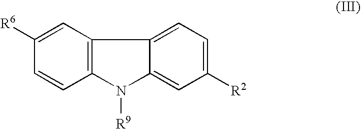

wherein R² is

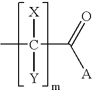

wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(—O)OR⁷ (where R⁷ is (C₁-C₂)alkyl);
or a pharmaceutically acceptable salt or solvate thereof,
together with a pharmaceutically acceptable excipient or carrier.

The combination of a compound of formula (I) or formula (II) with a compound of formula (III) may be synergistic.

In one embodiment of the invention the compound of formula (I) is gabapentin.

In one embodiment of the invention, in formula (II), R¹² and R¹³ are hydrogen and R¹¹ is —(CH₂)₀₋₂-iC₄H₉.

In another embodiment of the invention the compound of formula (II) is pregabalin.

In one embodiment of the invention the compound of formula (III) is carprofen.

In one embodiment of the invention the combination comprises gabapentin and carprofen. The combination may be synergistic.

In another embodiment of the invention the combination comprises pregabalin and carprofen. The combination may be synergistic.

In one embodiment of the invention the treatment is for dogs. In another embodiment the treatment is for cats. In a further embodiment the treatment is for horses.

In one embodiment of the invention the ratio of alpha-2-delta ligand of formula (I) or formula (II) to non-steroidal anti-inflammatory compound of formula (III) is from 100:1 to 1:1.

In another embodiment of the invention the ratio of alpha-2-delta ligand of formula (I) or formula (II) to non-steroidal anti-inflammatory compound of formula (III) is from 50:1 to 5:1.

In an alternative embodiment of the invention the ratio of alpha-2-delta ligand of formula (I) or formula (II) to non-steroidal anti-inflammatory compound of formula (III) is 9:1.

### BRIEF DESCRIPTION OF THE DRAWINGS

FIG. 1 shows the effect on paw withdrawal latency (seconds) after challenge with carrageenan and administration of a fixed dose ratio of 1:10 (1 part by weight/kg of carprofen to 10 parts by weight/kg of Gabapentin) carprofen:gabapentin using a total dose of 11 mg/kg in the rat carrageenan-induce thermal hyperalgesia model. Comparison is made to the predicted additive line for a 1:10 fixed dose ratio of carprofen:gabapentin.

### DETAILED DESCRIPTION OF THE INVENTION

The present invention relates to a combination comprising an alpha-2-delta ligand of formula (I)

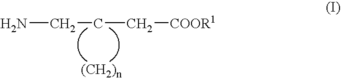

wherein R¹ is hydrogen or (C₁-C₄)alkyl; n is an integer of from 4 to 6; or a pharmaceutically acceptable salt or solvate thereof, or
an alpha-2-delta ligand of formula (II)

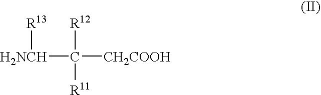

wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; and
a non-steroidal anti-inflammatory compound of formula (III)

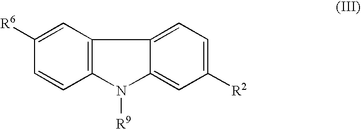

wherein R² is

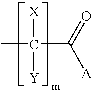

wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof.

The invention also relates to a pharmaceutical composition comprising a combination of an alpha-2-delta ligand, such as a compound of formula (I) or formula (II) as defined herein, and a non-steroidal anti-inflammatory compound of formula (III), as defined herein, together with a pharmaceutically acceptable excipient or carrier.

The combination of alpha-2-delta ligand and non-steroidal anti-inflammatory compound may be synergistic.

The invention also relates to the use of an alpha-2-delta ligand, such as a compound of formula (I) or formula (II) as defined herein, and a non-steroidal anti-inflammatory compound of formula (III), as defined herein, for the curative, prophylactic or palliative treatment of pain and/or inflammation in a mammal, in particular cats, dogs and horses.

Thus, the present invention also relates to the use of an alpha-2-delta ligand, such as a compound of formula (I) or formula (II) as defined herein, and a non-steroidal anti-inflammatory compound of formula (III), as defined herein, in the manufacture of a medicament for the curative, prophylactic or palliative treatment of pain and/or inflammation in a mammal selected from cats, dogs and horses.

The invention also relates to a method for the curative, prophylactic or palliative treatment of pain and/or inflammation in a mammal selected from cats, dogs and horses comprising administering to the mammal an effective amount of an alpha-2-delta ligand, such as a compound of formula (I) or formula (II) as defined herein, and a non-steroidal anti-inflammatory compound of formula (III), as defined herein.

The combination of an alpha-2-delta ligand, such as a compound of formula (I) or formula (II), and a non-steroidal anti-inflammatory compound of formula (III) may be synergistic. Thus, according to the present invention, combination therapy with an alpha-2-delta ligand, such as a compound of formula (I) or formula (II), and a non-steroidal anti-inflammatory compound of formula (III) results in an unexpected improvement in the veterinary treatment of pain and/or inflammation, particularly in dogs, cats and horses.

When administered simultaneously, sequentially or separately, the alpha-2-delta ligand of formula (I) or formula (II), and the non-steroidal anti-inflammatory compound of formula (III) interact in a synergistic manner to control pain and/or inflammation.

This unexpected synergy provides enhanced clinical efficacy compared to the individual components of the combination when administered separately, or a reduction in the required dose of each compound, leading to a reduction in side effects whilst maintaining or enhancing the clinical effectiveness of the compounds and treatment. For example, when administered simultaneously, sequentially or separately, the patient may experience an improved reduction in the frequency and severity of pain and/or inflammation. Furthermore, the patient may benefit from a longer duration of action from the combination treatment than from treatment with the either alpha-2-delta ligand of formula (I) or formula (II) alone, or the non-steroidal anti-inflammatory compound of formula (III) alone.

In another embodiment, in the non-steroidal anti-inflammatory compound of formula (III), A is hydroxy, X and Y are each independently selected from H and methyl; m is 1 or 2; R⁶ is halogen; and R⁹ is H or (C₁-C₂)alkyl.

In another embodiment, the non-steroidal anti-inflammatory compound of formula (III) exists as (R) and (S) enantiomers. In one embodiment the combination comprises a mixture of the (R) and (S) enantiomers. In another embodiment the combination comprises the (S) enantiomer but not the (R) enantiomer.

In a further embodiment, the non-steroidal anti-inflammatory compound of formula (III) is carprofen. In another embodiment the non-steroidal anti-inflammatory compound of formula (III) is (S)-carprofen.

Examples of alpha-2-delta ligands suitable for use in accordance with the present invention are those compounds generally or specifically disclosed in U.S. Pat. No. 4,024,175, particularly gabapentin, EP641330, particularly pregabalin, U.S. Pat. No. 5,563,175, WO 97/33858, WO 97/33859, WO 99/31057, WO 99/31074, WO 97/29101, WO 02/085839, particularly [(1R,5R,6S)-6-(aminomethyl)-bicyclo[3.2.0]hept-6-yl]acetic acid, WO 99/21824, particularly (3S,4S)-(1-aminomethyl-3,4-dimethyl-cyclopentyl)-acetic acid, WO 01/90052, WO 01/28978, particularly (1α,3α,5α)(3-amino-methyl-bicyclo[3.2.0]hept-3-yl)-acetic acid, EP0641330, WO 98/17627, WO 00/76958, particularly (3S,5R)-3-aminomethyl-5-methyl-octanoic acid, WO 03/082807, particularly (3S,5R)-3-amino-5-methyl-heptanoic acid, (3S,5R)-3-amino-5-methyl-nonanoic acid and (3S,5R)-3-amino-5-methyl-octanoic acid, and pharmaceutically acceptable salts and solvates thereof.

In one embodiment, in the compound of formula (II) R¹² and R¹³ are hydrogen, and R¹¹ is —(CH₂)₀₋₂-iC₄H₉. In another embodiment the alpha-2-delta ligand is pregabalin.

In another embodiment the alpha-2-delta ligand is gabapentin.

In one embodiment the composition comprises carprofen, in particular (S)-carprofen, and gabapentin. In another embodiment the composition comprises carprofen, in particular (S)-carprofen, and pregabalin.

The combination according to the present invention may be prepared as a single dosage form suitable for administration to cats, dogs, or horses.

In one embodiment administration is once, twice or three times daily. In another embodiment, administration is twice or three times daily. In a further embodiment administration is twice daily. In another embodiment administration is once daily. Alternatively, administration may be once, twice or three times weekly. In another embodiment administration may be twice or three times weekly. In a further embodiment administration may be twice weekly. In another embodiment administration may be once weekly.

In another embodiment of the present invention, there is provided a synergistic combination suitable for veterinary administration comprising an alpha-2-delta ligand of formula (I) or formula (II) and an anti-inflammatory agent of formula (III), as defined herein, or pharmaceutically acceptable salts or solvates thereof, in a w/w combination range selected from between 1:50 to 50:1 parts by weight, 1:50 to 20:1, 1:50 to 10:1, 1:50 to 1:1, 1:20 to 50:1, 1:20 to 20:1, 1:20 to 10:1, 1:20 to 1:1, 1:10 to 50:1, 1:10 to 20:1, 1:10 to 10:1, 1:10 to 1:1, 1:1 to 50:1, 1.1 to 20:1 and 1:1 to 10:1 parts by weight, more suitably 1:20 to 20:1 parts by weight, even more suitably, 1:20 to 1:1 parts by weight, respectively.

In one embodiment, a suitable dose ratio for administration of a compound of formula (I) or formula (II) in combination with a compound formula (III) is a ratio of 9:1, respectively.

Compounds of formula (III), including carprofen, utilized in the methods and compositions of the present invention may be prepared in accordance with methods of synthesis previously published and well known to the organic chemist of ordinary skill. For example, synthetic methods for the preparation of compounds of formula (III), including carprofen, which may be utilized in the methods and compositions of the present invention are described in detail in U.S. Pat. No. 3,896,145.

In the definition of the ‘R²’ substituent of compounds of formula (III), when ‘X’ and ‘Y’ are different, a chiral (asymmetric) carbon atom exists. The stereochemistry at the asymmetric carbon atom may be (R) or (S). A racemic mixture of (R)- and (S)-enantiomers results when there is a 50:50 mixture of the two enantiomers. The present invention disclosed herein includes all enantiomeric, diastereomeric and epimeric forms of the compounds of formula (III), as well as mixtures thereof.

The (S)-enantiomer of the carprofen genus of compounds of formula (III) having a chiral carbon is the enantiomer which possesses the highest level of activity in treating or preventing degeneration or destruction of the articular cartilage or subchondral bone of a mammal subject identified as being in the early stages of articular cartilage degeneration which eventually results in injury or loss of cartilage or subchondral bone in any involved joints thereof. Accordingly, in one embodiment of the invention the compound of formula (III) is (S)-carprofen.

One especially preferred embodiment of the present invention is to use the (S)-enantiomer of carprofen, (S)-6-chloro-α-methyl-9H-carbazole-2-acetic acid, as the compound of formula (III) in the methods and combinations of the present invention. However, other embodiments are contemplated to be within the scope of the present invention as well. For example, non-racemic mixtures of the (R)- and (S)-enantiomers may be used, and in that event the (S)-enantiomer may be present in an amount, with respect to the total amount of the compound formula (III) in the combination, of at least 85%, preferably at least 90%, more preferably at least 95%, and most preferably at least 99%. Since the (R)- and (S)-enantiomers are identical in molecular weight, density, etc., it is unnecessary to state any basis for the above-recited percentages. In other words, they could be percentages by weight, volume, chemical equivalency, etc. The reason for including the above-indicated amounts of the (R)-enantiomer may be as simple as the practicalities of not being required to remove absolutely every last trace of the (R)-enantiomer from the racemic mixture. There can also be reasons for doing so which relate to beneficial overall biological properties. In such non-racemic mixtures the (+)(S) enantiomer will be the predominant component, because it is significantly more potent than the (−)(R) enantiomer. Correspondingly smaller amounts of the (−)(R) enantiomer, i.e., less than 15%, less than 10%, less than 5%, and less than 1% respectively, optionally may be included in combinations in accordance with the invention.

It will also be appreciated by those in the art that the ranges of dosage amounts recited elsewhere herein for the formula (III) compounds of the combination are being described with respect to a 50:50 racemic mixture of enantiomers, where a chiral compound is involved. This has been done largely as a matter of convenience. Where the formula (III) compound comprises a mixture of enantiomers different from a 50:50 mixture, or where the formula (III) compound comprises substantially 100% of the (+)(S) or (−)(R) enantiomer alone, the person of ordinary skill in this art will be able to calculate the actual amount of dosage required in a very straightforward manner, simply by multiplying the dosage amounts recited by a factor which reflects the ratio of the amount of enantiomer being used to the amount present for the recited dosage based on a 50:50 mixture of the enantiomers. Accordingly, where a dosage would be 4 mg/kg/day for the 50:50 racemic mixture, the corresponding dosage amount when substantially 100% of (+)(S) enantiomer is used one-half of the recited amount, i.e., 2 mg/kg/day.

The alpha-2-delta ligands of formula (I) and formula (II) may be prepared using previously published methods, including for example the methods described in U.S. Pat. No. 4,024,175 and EP641330. Certain compounds of formula (I) or formula (II) possess one or more chiral centres and each centre may exist in the (R) or (S) configuration. The present invention includes all enantiomeric, diastereomeric and epimeric forms of the compounds of formulae (I) and (II), as well as mixtures thereof.

The compounds of the present combination invention can exist in unsolvated forms as well as solvated forms, including hydrated forms. In general, the solvated forms, including hydrated forms, which may contain isotopic substitutions (e.g. D₂O, d6-acetone, d6-DMSO), are equivalent to unsolvated forms and are encompassed within the scope of the present invention.

Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof.

A number of the compounds in the combination of the present invention are amino acids. Since amino acids are amphoteric, pharmacologically compatible salts can be salts of appropriate non-toxic inorganic or organic acids or bases. Suitable acid addition salts are the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts and examples are the sodium, potassium, aluminium, calcium, magnesium, zinc, choline, diolamine, olamine, arginine, glycine, tromethamine, benzathine, lysine, meglumine and diethylamine salts. Salts with quaternary ammonium ions can also be prepared with, for example, the tetramethyl-ammonium ion. The compounds of the invention may be zwitterionic.

A suitable salt for amino acid compounds of the present invention is the hydrochloride salt. For a review on suitable salts see Stahl and Wermuth, Handbook of Pharmaceutical Salts: Properties, Selection, and Use, Wiley-VCH, Weinheim, Germany (2002).

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds in the combination of the invention include references to salts thereof and to solvates and clathrates of compounds of the invention and salts thereof.

Also included within the present scope of the invention are polymorphs of the compounds comprised by the combination.

Prodrugs of the above compounds of the invention are included in the scope of the instant invention. The chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be
(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.
(2) Peptides which may be recognized by specific or nonspecific proteinases. A peptide may be coupled to the drug molecule via amide bond formation with the amine or carboxylic acid moiety of the drug molecule by known means.
(3) Derivatives that accumulate at a site of action through membrane selection of a prodrug form or modified prodrug form.
(4) Any combination of (1) to (3).

Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990; 15(4):361-368).

The combination according to the present invention optionally may be administered with one or more other pharmacologically active agents. Suitable optional agents include glucosamine, chondroitin, hyaluronic acid, steroids, opioids, tetracycline, IL-1 release inhibitor and diacerhein.

The active ingredients of the present invention may also be combined with other therapeutically active ingredients which would be readily apparent to the skilled artisan in this field, and which will usually be determined by the circumstances under which the therapeutic agent of the present invention is administered. For example, where a joint has become seriously infected at the same time by microorganisms, e.g., bacteria, fungi, protozoa, virus and the like, the active ingredient of the present invention will desirably be administered in combination with one or more antibiotic, antifungal, antiprotozoal, antiviral or similar therapeutic agents. The active ingredients of the present invention may be administered in combination with other NSAIDs as well as with inhibitors of other mediators of inflammation. Additional classes of such inhibitors and examples thereof include, e.g., H1-receptor antagonists; kinin-B1- and B2-receptor antagonists; prostaglandin inhibitors such as PGD-, PGF- PGI2-, and PGE-receptor antagonists; thromboxane ˜(TXA2-) inhibitors; 5- and 12-lipoxygenase inhibitors; leukotriene LTC4-, LTD4/LTE4-, and LTB4-inhibitors; PAF-receptor antagonists; gold in the form of an aurothio group together with various hydrophilic groups; immunosuppressive agents, e.g., cyclosporine, azathioprine, and methotrexate; anti-inflammatory glucocorticoids, e.g., dexamethasone; broad-spectrum antiparasitic antibiotics, e.g., the avermectins and the milbemycins; penicillamine; hydroxychloroquine; anti-gout agents, e.g., colchicine, xanthine oxidase inhibitors, e.g., allopurinol, and uricosuric agents, e.g., probenecid, sulfinpyrazone, and benzbromarone.

The class of therapeutic agents which are broad-spectrum antiparasitic antibiotics, e.g., the avermectins and the milbemycins, are especially good candidates for co-administration and other types of combination therapy with the compounds of formula (I) and formula (III), or formula (II) and formula (III) since these endo- and ecto-parasiticides are administered on a chronic basis to mammals, especially to cats and dogs for the treatment of serious parasitic infestations. One of the most significant of these is heartworm, which is a very damaging and often fatal parasitic affliction of cats and dogs. The avermectins are a class of pentacyclic 16-membered lactones related in structure to the milbemycins, and are isolated from cultures of Streptomyces avermitilis. Specific agents include avermectin A1 a/b′, avermectin A2a/b′, avermectin B1 a/b′, and avermectin B2a/b″. The avermectins are described in more detail in U.S. Pat. No. 4,310,159, which is incorporated herein by reference in its entirety. The milbemycins are a family of novel macrolide antibiotics with insecticidal and acaricidal activity, and are isolated from cultures of Streptomyces hygroscopicus. The milbemycins are described in more detail in U.S. Pat. No. 3,950,360, which is incorporated herein by reference in its entirety. Yet another family of compounds included within the scope of the broad-spectrum antiparisitic antibiotics, is one related in chemical structure and biological activity to the avermectins and the milbemycins.

Some macrolides are described in more detail in WO 94/15944 and EP 0677054.

Because the early stages of articular cartilage degeneration are prevalent among geriatric companion animals, compounds of formula (I) and (III) or (II) and (III) may also be administered in combination with therapeutic agents intended for the treatment of disease conditions, syndromes and symptoms which are also found in abundance in older companion animals. Such therapeutic agents and the conditions which they are used to treat include, e.g., cognitive therapeutics to counteract memory loss and impairment; and antidyskinetic/antiparkinsonian agents, e.g., selegeline, clomipramine. Another large class of such therapeutic agents includes anti-hypertensives and other cardiovascular drugs intended to offset hypertension, myocardial ischemia including angina, congestive heart failure, and myocardial infarction, e.g., diuretics, vasodilators such as hydralazine, α-adrenergic receptor antagonists such as propranolol, angiotensin-II converting enzyme inhibitors (ACE-inhibitors) such as enalapril or benazepril used to treat geriatric companion animals with mitral insufficiency, and enalapril alone and in combination with neutral endopeptidase inhibitors, angiotensin II receptor antagonists such as losartan, renin inhibitors, calcium channel blockers such as nifedipine, diffingen, oranlodipine, sympatholytic agents such as methyldopa, α2-adrenergic agonists such as clonidine, α-adrenergic receptor antagonists such as prazosin, and HMG-CoA-reductase inhibitors (anti-hypercholesterolemics) such as lovastatin.

Still other classes of such therapeutic agents include antineoplastic agents, especially antimitotic drugs including the vinca alkaloids such as vinblastine and vincristine, for treating various cancers; therapeutic agents for treating renal failure; anti-obesity drugs for treating excess weight problems in companion animals; anti-parasitic drugs for treating both endo- and ecto-parasites which commonly afflict companion animals; and anti-pruritic drugs for treating various types of pruritus in companion animals.

Other types of drugs which can be used in combination with the compounds of formula (I) and formula (III), or formula (II) and formula (III) of the present invention include growth hormone secretagogues; strong analgesics; local and systemic anaesthetics; and H₂-receptor antagonists and other gastroprotective agents. It will be recognized by those of ordinary skill in this art that some of the above combinations of therapeutic agents will be used most frequently to treat various acute conditions in companion animals, e.g., bacterial infections occurring simultaneously with degenerative joint disease. However, there would be an equal if not greater interest on the part of such skilled persons in treating chronic conditions in companion animals, such as cats, dogs and horses.

In accordance with a regimen which would be used for this purpose, it is contemplated that the compounds of formula (I) and formula (III), or formula (II) and formula (III) would be administered in combination with other medications used on a regularly scheduled basis for treating chronic conditions such as hyperlipidemia. It is also envisaged that administration in combinations could assume a number of different forms and still be within the scope of the present invention. For example, the compounds of formula (III) may be formulated with one or more of the other therapeutic agents which are to form the intended combination, into a convenient dosage form, such as an oral tablet, containing all of the drugs forming the combination. Varying half-lives for the different drugs could be accommodated by the person skilled in preparing formulations by creating controlled-release forms of said drugs with different release times so that relatively uniform dosing was achieved. A medicated feed used as the dosage form could also be prepared in accordance with well known principles in the art of formulation, in which the drugs used in the combination were simply present together in admixture in the feed composition. The present invention also contemplates co-administration in which the combination of drugs is achieved by the simultaneous administration of the drugs to be given in combination. Such co-administration could even be by means of different dosage forms and routes of administration. The present invention further contemplates the use of such combinations in accordance with different but regular and continuous dosing schedules whereby desired plasma levels of the drugs involved were maintained in the companion animal being treated, even though the individual drugs making up the combination were not being administered to said companion animal simultaneously. All such combinations would be well within the skill of the art to devise and administer.

Thus, the present invention extends to a combination product as described hereinbefore comprising one or more additional therapeutic agents, such as one of those listed above, for simultaneous, separate or sequential use in the curative, prophylactic or palliative treatment of pain and/or inflammation.

The combination according to the present invention is useful for the general treatment of pain, particularly inflammatory pain.

Although patients with pain after surgery, joint pain, osteoarthritis, degenerative joint disease, back pain, arthritis pain, CNS trauma, or neuropathic pain may have similar symptoms, the underlying mechanisms can be different and various. Therefore pain can be divided into a number of different types because of differing pathophysiology, these include nociceptive, inflammatory, neuropathic pain etc. It should be noted that pain can have multiple aetiologies and thus a complex combination of pain types may co-exist and the contribution of different pain types may vary with time.

The inflammatory process is a complex series of biochemical and cellular events activated in response to tissue injury or the presence of foreign substances, which result in swelling and pain (Levine and Taiwo 1994: Textbook of Pain, 45-56). Arthritic pain makes up the majority of the inflammatory pain population. Osteoarthritis and degenerative joint disease are the most common clinical problems in dogs, cats and horses many receive medical treatment. Current treatments include NSAIDs, steroids, nutraceuticals and glycosaminoglycans. Rheumatoid disease can occur in cats and dogs. The exact aetiology of rheumatoid arthritis is unknown, but current hypotheses suggest that both genetic and microbiological factors may be important (Grennan & Jayson 1994 Textbook of Pain 397-407). Arthritis has a significant impact on physical function and is known to be the leading cause of disability in later life.

For veterinary use, a combination according to the present invention is administered as a suitably acceptable formulation in accordance with normal veterinary practice. The veterinary surgeon will determine the dosing regimen and route of administration, which will be most appropriate for a particular animal.

The combination of the invention can be administered alone but one or both elements will generally be administered in an admixture with suitable pharmaceutical excipient(s), diluent(s) or carrier(s) selected with regard to the intended route of administration and standard pharmaceutical practice. If appropriate, auxiliaries can be added. Auxiliaries are preservatives, anti-oxidants, flavours or colourants. The compounds of the invention may be of immediate-, delayed-, modified-, sustained-, pulsed- or controlled-release type.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The methods by which the compounds may be administered include oral administration by capsule, cachet, caplet bolus, tablet, chewable tablet, powders, lozenges, chews, multi and nanoparticulates, gels, solid solution, films, sprays, or liquid formulation. Liquid forms include suspensions, solutions, syrups, drenches and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. Oral drenches are commonly prepared by dissolving or suspending the active ingredient in a suitable medium.

As an alternative the compounds may be administered with the feedstuff and for this purpose a concentrated feed additive or premix may be prepared for mixing with the normal animal feed.

Other pharmaceutical compositions of special types suitable for oral administration to companion animals may be used, and include, but are not limited to such items as an oral paste to be delivered to the back of the tongue of the companion animal being treated, a granular form to be delivered through incorporation in the companion animal's food, and a chewable form wherein the active ingredient is consumed along with the palatable chew, or a chewable form which may deliver the active ingredient by leaching from the body of the chew which is not consumed, during mastication by the companion animal being treated. As is known in the art, the formulation of such palatable compositions takes into account companion animal behaviour regarding the extent of mastication of the dosage form which will take place, and the resultant level of dosing.

Thus, compositions useful for oral administration may be prepared by mixing the active ingredient with a suitable finely divided diluent and/or disintegrating agent and/or binder, and/or lubricant etc. Other possible ingredients include anti-oxidants, colorants, flavouring agents, preservatives and taste-masking agents.

For oral dosage forms, depending on dose, the drug may make up from 1 wt % to 80 wt % of the dosage form, more typically from 5 wt % to 60 wt % of the dosage form. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Examples of diluents include lactose (monohydrate, spray-dried monohydrate, anhydrous and the like), mannitol, xylitol, dextrose, sucrose, sorbitol, microcrystalline cellulose, starch and dibasic calcium phosphate dihydrate.

Oral formulations may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Lubricants include magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The compounds may be administered topically to the skin or mucosa, that is dermally, epidermally, subepidermally or transdermally. Typical formulations for this purpose include pour-on, spot-on, dip, spray, mousse, shampoo, powder formulation, gels, hydrogels, lotions, solutions, creams, ointments, dusting powders, dressings, foams, films, skin patches, wafers, implants, depots, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999). Pour-on or spot-on formulations may be prepared by dissolving the active ingredient in an acceptable liquid carrier vehicle such as butyl digol, liquid paraffin or a non-volatile ester, optionally with the addition of a volatile component such as propan-2-ol. Alternatively, pour-on, spot-on or spray formulations can be prepared by encapsulation, to leave a residue of active agent on the surface of the animal. Injectable formulations may be prepared in the form of a sterile solution which may contain other substances, for example enough salts or glucose to make the solution isotonic with blood. Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending the active ingredient in the liquid carrier such that the final formulation contains from 0.01 to 10% by weight of the active ingredient.

Alternatively, the compounds can be administered parenterally, by injection directly into the blood stream, muscle or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as powdered a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of compounds of formula (I), (II) or (III) used in the preparation of parenteral solutions may be increased by the use of appropriate formulation techniques, such as the incorporation of solubility-enhancing agents.

Salt forms of the compounds in the combination of the present invention may be required for reasonable good aqueous solubility. If it is not possible to obtain a form of the compounds in the present combination that have the requisite degree of aqueous solubility, it is within the skill of the artisan to prepare an emulsion, which is a dispersion of small globules of one liquid, the discontinuous or internal phase, throughout a second liquid, the continuous or external phase, with which it is immiscible. The two liquids are maintained in an emulsified state by the use of emulsifiers which are pharmaceutically acceptable. Thus, if a compound in the combination of the present invention is a water-insoluble oil, it can be administered in an emulsion of which it is the discontinuous phase. Also, where the compound is water-insoluble but can be dissolved in a solvent which is immiscible with water, an emulsion can be used. While the compound of formula (III) would commonly be used as the discontinuous or internal phase of what is referred to as an oil-in-water emulsion, it could also be used as the discontinuous or internal phase of an inverse emulsion, which is commonly referred to as a water-in-oil emulsion. In this instance the compound of formula (III) is soluble in water and could be administered as a simple aqueous solution.

However, inverse emulsions invert upon injection or infusion into an aqueous medium such as the blood, and offer the advantage of providing a more rapid and efficient dispersion of said compound into that aqueous medium than can be obtained using an aqueous solution. Inverse emulsions are prepared by using suitable, pharmaceutically acceptable emulsifying agents well known in the art. Where the compound in the combination of the present invention has limited water solubility, it may also be administered as a suspended solid in colloidal or microparticulate form in a suspension prepared using suitable, pharmaceutically acceptable suspending agents. The suspended solids containing said compound may also be formulated as delayed-, sustained-, and/or controlled-release compositions.

There are many situations in which it will be advantageous or even necessary to deliver the combination of the present invention as a solid. Systemic administration of solids is carried out by instillation of a pharmaceutical composition in suitable solid form containing said compound. Instillation of said compound may entail installing a solid implant composition into suitable body tissues or cavities. The implant may comprise a matrix of bio-compatible and bioerodible materials in which particles of the combination of the present invention are dispersed, or in which, possibly, globules or isolated cells of a liquid mixture of the present combination are entrapped. Desirably, the matrix will be broken down and completely absorbed by the body. The composition of the matrix is also preferably selected to provide controlled-, sustained-, and/or delayed release of the combination of the present invention over extended periods of 30 time, even as much as several months.

Such formulations may be prepared in a conventional manner in accordance with, standard veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are from 0.1 to 100 mg per kg of body weight of the animal. Preferably the range is from 0.5 to 50 mg per kg. A suitable dose for administration to a dog is 20 mg/kg of an alpha-2-delta ligand of formula (I) or (II), such as gabapentin or pregabalin, with 2.2.mg/kg of a compound of formula (III), such as carprofen. In one embodiment, the combination suitably may be administered twice daily for 14 days.

Formulations may be immediate and/or modified controlled release. Controlled release formulations include Modified release formulations including delayed-, sustained-, pulsed-, controlled-, targeted, or programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298. Alternatively, compounds of the invention may be formulated as a solid, semi-solid, or thixotropic liquid for administration as an implanted depot providing modified release of the active compound. Examples of such formulations include drug-coated stents and PGLA microspheres.

As an alternative the compounds may be administered with the feedstuff and for this purpose a concentrated feed additive or premix may be prepared for mixing with the normal animal feed. Systemic administration can also be accomplished by inhalation or insufflation of a powder, i.e., particulate composition containing the inhibitor. For example, the inhibitor in powder form may be inhaled into the lungs using conventional devices for aerosolising particulate formulations. The inhibitor as a particulate formulation may also be administered by insufflation, i.e., blown or otherwise dispersed into suitable body tissues or cavities by simple dusting or using conventional devices for aerosolizing particulate formulations.

The elements of the combination of the invention may also be used in combination with a cyclodextrin.

It is necessary for the skilled artisan, such as a veterinarian, not only to determine the preferred route of administration and the corresponding dosage form and amount, but said artisan must also determine the dosing regimen, i.e., the frequency of dosing. In general terms it is most likely that the choice will be between once-a-day (q.d.) dosing and twice-a-day (b.i.d.) dosing, and that the former will provide more rapid and profound therapy, while the latter will provide less profound but more sustained therapy. However, this generalization does not take into account such important variables as the specific type of articular cartilage or subchondral bone degeneration or destruction involved, the specific therapeutic agent involved and its pharmacokinetics, and the specific patient (companion animal) involved. For an approved product in the marketplace, much of this information is already provided by the results of clinical studies carried out to obtain such approval. In other cases, such information may be obtained in a straightforward manner in accordance with the teachings and guidelines contained in the instant specification taken in light of the knowledge and skill of the artisan. The results which are obtained can also be correlated with data from corresponding evaluations of an approved product in the same assays.

As an alternative aspect of the present invention, there is provided a pharmaceutical composition comprising a compound of formula (I) or (II) in combination with a compound of formula (III), as defined herein, or a combination of preferred features thereof, and a suitable excipient, diluent or carrier. The combination may be synergistic. Suitably, the composition is suitable for use in the treatment of pain and/or inflammation in mammals, in particular, a mammal selected from dogs, cats and horses.

The element of the pharmaceutical preparation is preferably in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsules, tablet, cachet, caplets, chews, in feed, in drink or lozenge itself, or it can be the appropriate number of any of these in packaged form. The quantity of active component in a unit dose preparation may be varied or adjusted according to the particular application and the potency of the active components. Generally, treatment is initiated with smaller dosages which are less than the optimum dose of the compounds. Thereafter, the dosage is increased by small increments until the optimum effect under the circumstances is reached. For convenience, the total daily dosage may be divided and administered in portions during the day, if desired. Alternatively, a starting dose to achieve therapeutic efficacy can be titrated down until a minimum therapeutic dose is established in the individual patient.

The combination according to the present invention may be in the form of a kit. Therefore, as a further aspect of the present invention, there is provided a kit for the curative, prophylactic or palliative treatment of pain and/or inflammation in cats, dogs or horses, comprising:

a) an alpha-2-delta ligand of formula (I) or formula (II), and
b) a non-steroidal anti-inflammatory compound of formula (III), wherein each of a) and b) are present in an effective amount, optionally comprising one or more pharmaceutically acceptable carriers, excipients or diluents, and
wherein a) and b), may be present in the same formulation or in separate formulations, suitable for simultaneous, sequential or separate administration, and
c) packaging for containing a) and b)

### BIOLOGICAL EXAMPLES AND DATA

The in vitro and ex vivo COX activity of compounds of formula (III) can be determined using the assays described in WO 98/50033 and other references well known to those skilled in the art.

Evaluation of alpha-2-delta Activity

The biological activity of the alpha-2-delta ligands of the invention may be measured in a radioligand binding assay using [³H]gabapentin and the α₂ δ subunit derived from porcine brain tissue (Gee N. S., Brown J. P., Dissanayake V. U. K., Offord J., Thurlow R., Woodruff G. N., J. Biol. Chem., 1996; 271:5879-5776). Results may be expressed in terms of μM or nM α2 δ binding affinity.

There are numerous rodent models cited in the literature that demonstrate the effect of analgesic compounds. These models are considered to be representative of different pain types such as nociception, inflammatory or neuropathic pain, as well as hyperalgesia and allodynia. However, in diseases of dogs, cats and horses, like osteoarthritis, or after surgery, a complex combination of pain types may coexist and the contribution of individual pain types vary with time.

Determining a synergistic interaction between one or more components, the optimum range for the effect and absolute dose ranges of each component for the effect may be definitively measured by administration of the components over different doses to patients in need of treatment.

### Rat Data:

### EXAMPLE 1

The aim of this experiment was to characterize the anti-nociceptive and anti-inflammatory effects of gabapentin administered in combination with carprofen in the rat. In this example, gabapentin, carprofen, and the combination of gabapentin and carprofen were evaluated in a standard rat carrageenan footpad thermal hyperalgesia assay. This assay utilizes an extract of seaweed (carrageenan) that, when injected into the footpad of test animals, causes a sterile inflammation, thereby lowering the pain threshold. Anti-epileptic agents having analgesic properties, such as gabapentin, raise the pain threshold back to normal, thereby enabling the animal to tolerate an external source of pain for a longer period of time relative to untreated control animals.

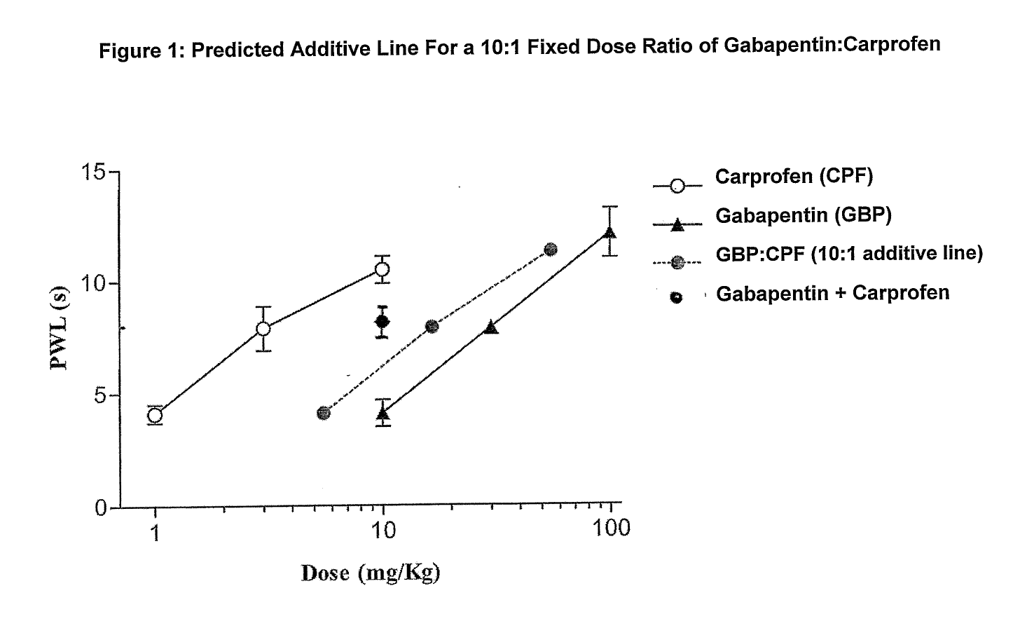

As shown in FIG. 1, gabapentin and carprofen were given alone (gabapentin at 120 min after dosing; carprofen at 120 min after dosing). Each data point represents the mean and standard error of mean. Data for each drug were fitted by least squares linear regression to a straight line. The theoretical dose-additive line for a 10:1 dose ratio was determined (dotted line) as described (Tallarida, 1992). The experimental determination of a 10:1 dose ratio was determined (gabapentin-carprofen mixture 10:1) and a shift to the left of the theoretical dose-additive line was found. Thus, a supra-additive effect was determined for the combination of the two treatments given simultaneously.

To summarize, the data shows that both gabapentin (10-100 mg/kg SC) and carprofen (1-10 mg/kg SC) cause antihyperalgesic actions in the rat carrageenan footpad model (Hargreaves test). Combinations in a fixed ratio (10 mg gabapentin/1 mg carprofen or 10:1 ratio) were antihyperalgesic, and produced a significantly supra-additive effect (synergistic action). For example, with a 10:1 dose ratio, dosages of carprofen (1 mg/kg) plus gabapentin (10 mg/kg) that both caused an effect of approximately 10% or less than the respective compounds alone, produced marked antihyperalgesic effects of approximately 65% when given in combination.

The data establish that the combination of gabapentin and carprofen is synergistic in its ability to relieve acute and chronic pain. The data also establish that the most preferred combination of gabapentin plus carprofen is in a fixed-ratio combination preferably from 50:1 to 1:50, more preferably from 20:1 to 1:20.

### Methods

### Animals

Male CD1 rats (175-225 g, Charles River) were used. Rats were acclimated for at least 5 days with free access to food and water. Rats received only one dose of a drug or drug combination. All drugs were administered subcutaneously by injection.

### Experimental Design

Dose-effect curves were first determined for (1) gabapentin by itself and (2) carprofen by itself. The ED50 value of each agent was determined, as was the time to peak effect. After determination of these values, dose effect curves were generated for gabapentin administered in a fixed dose ratio with carprofen; the drugs were administered so that their peak effects were coincident.

### Measures of Antinociception

Carrageenan-induced thermal hyperalgesia: Rats were acclimated to a testing chamber whose glass floor was maintained at 25° C. One hour later, a high intensity beam of light was focused through the glass on the ventral surface of each hind paw, and the latency to reflex withdrawal of the paw from the light beam was measured to the nearest 0.1 second. This latency was termed the paw withdrawal latency (PWL). Two measurements of PWL spaced 20 minutes apart were made for each paw, and the second measurement was taken as the baseline response latency. After determination of baseline PWL, 100 μL of 20 mg/ml carrageenan was injected in the plantar surface of one hind paw and the animal returned to the testing chamber. Two hours later, when thermal hyperalgesia was maximal and stable, either vehicle, gabapentin, carprofen, or gabapentin and carprofen was administered by gavage. Response latencies for the ipsilateral and contralateral hind paws were then re-determined 60, 120 and 180 minutes later. Data for further analysis were taken 60 minutes after subcutaneous dosing.

### Statistical Analysis

Data were expressed as the mean ±SEM. Two-way analyses of variance for repeated measures were used to compare the effects of drug to that of vehicle. Dose-effect lines for gabapentin and the NSAID were constructed using individual data and fitted with least squares linear regression analysis to determine ED50 values. A similar analysis was conducted for the drugs in combination using the total dose administered. Since parallel dose-effect lines were obtained for gabapentin and carprofen then the position of the experimentally-derived dose-effects for the combination were compared to the position of the theoretical dose-additive line. A shift to the left or the right of the theoretical dose-additive line indicates that the drugs interacted in a supra-additive (synergistic) or an infra-additive manner (antagonistic), respectively.

1. Use of an alpha-2-delta ligand of formula (I) 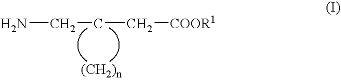
wherein R¹ is hydrogen or (C₁-C₄)alkyl; n is an integer of from 4 to 6; or a pharmaceutically acceptable salt or solvate thereof, or an alpha-2-delta ligand of formula (II) 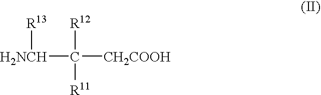
wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; and
a non-steroidal anti-inflammatory compound of formula (III) 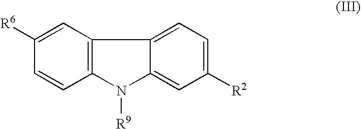
wherein R² is 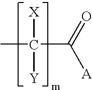
wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof,
in the manufacture of a medicament for the curative, prophylactic or palliative treatment of pain and/or inflammation in a mammal selected from cats, dogs and horses.

2. Use according to claim 1, wherein the compound of formula (III) is carprofen.

3. Use according to claim 1 or 2, wherein the compound of formula (I) is gabapentin.

4. Use according to claim 1 or 2, wherein in formula (II), R¹² and R¹³ are hydrogen and R¹¹ is —(CH₂)₀₋₂-iC₄H₉.

5. Use according to claim 4, wherein the compound of formula (II) is pregabalin.

6. Use according to any one of claims 1 to 5, wherein the treatment is for dogs.

7. A method for the curative, prophylactic or palliative treatment of pain and/or inflammation in a mammal selected from cats, dogs and horses comprising administering to the mammal an effective amount of an alpha-2-delta ligand of formula (I) 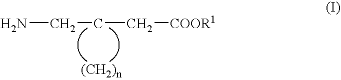
wherein R¹ is hydrogen or (C₁-C₄)alkyl; n is an integer of from 4 to 6; or a pharmaceutically acceptable salt or solvate thereof, or an alpha-2-delta ligand of formula (II) 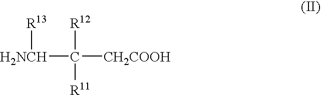
wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; and
a non-steroidal anti-inflammatory compound of formula (III), 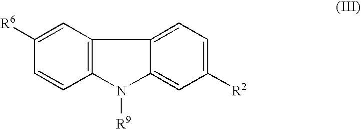
wherein R² is 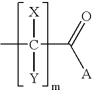
wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof.

8. The method according to claim 7, wherein the compound of formula (III) is carprofen.

9. The method according to claim 7 or 8, wherein the compound of formula (I) is gabapentin.

10. The method according to claim 7 or 8, wherein in formula (II), R¹² and R¹³ are hydrogen and R¹¹ is —(CH₂)₀₋₂-iC₄H₉.

11. The method according to claim 10, wherein the compound of formula (II) is pregabalin.

12. The method according to any one of claims 7 to 11, wherein the treatment is for dogs.

13. A pharmaceutical composition comprising an alpha-2-delta ligand of formula (I) 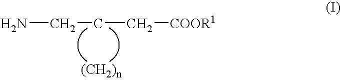
wherein R¹ is hydrogen or (C₁-C₄)alkyl; n is an integer of from 4 to 6; or a pharmaceutically acceptable salt or solvate thereof, or an alpha-2-delta ligand of formula (II) 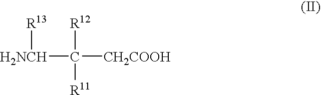
wherein R¹¹ is a straight or branched (C₁-C₆)alkyl, phenyl, or (C₃-C₆)cycloalkyl; R¹² is hydrogen or methyl; and R¹³ is hydrogen, methyl, or carboxyl; or an individual diastereomeric or enantiomeric isomer thereof; or a pharmaceutically acceptable salt or solvate thereof; and
a non-steroidal anti-inflammatory compound of formula (III) 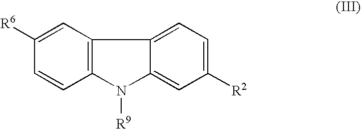
wherein R² is 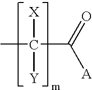
wherein A is hydroxy, (C₁-C₄) alkoxy, amino, hydroxyamino, mono-(C₁-C₂)alkylamino, di-(C₁-C₂)alkylamino;
X and Y independently are H or (C₁-C₂)alkyl;
m is 1 or 2;
R⁶ is halogen, (C₁-C₃)alkyl, trifluoromethyl, or nitro;
R⁹ is H, (C₁-C₂)alkyl, phenyl or phenyl(C₁-C₂)alkyl, (wherein phenyl, or the phenyl group in phenyl(C₁-C₂)alkyl, is optionally mono-substituted by fluoro or chloro), —C(═O)R (where R is (C₁-C₂)alkyl or phenyl, the R group being optionally mono-substituted by fluoro or chloro), —C(═O)OR⁷ (where R⁷ is (C₁-C₂)alkyl);
or a pharmaceutically acceptable salt or solvate thereof, together with a pharmaceutically acceptable excipient or carrier.

14. A pharmaceutical composition according to claim 13, wherein the ratio of alpha-2-delta ligand of formula (I) or formula (II) to non-steroidal anti-inflammatory compound of formula (III) is from 100:1 to 1:1.

15. A pharmaceutical composition according to claim 14, wherein the ratio of alpha-2-delta ligand of formula (I) or formula (II) to non-steroidal anti-inflammatory compound of formula (III) is from 50:1 to 5:1.

16. A pharmaceutical composition according to claim 15, wherein the ratio of alpha-2-delta ligand of formula (I) or formula (II) to non-steroidal anti-inflammatory compound of formula (III) is 9:1.

17. A pharmaceutical composition according to any one of claims 13 to 16, wherein the compound of formula (III) is carprofen.

18. A pharmaceutical composition according to any one of claims 13 to 17, wherein the compound of formula (I) is gabapentin.

19. A pharmaceutical composition according to any one of claims 13 to 17, wherein in formula (II), R¹² and R¹³ are hydrogen and R¹¹ is —(CH₂)₀₋₂-iC₄H₉.

20. A pharmaceutical composition according to claim 19, wherein the compound of formula (II) is pregabalin.

21. A pharmaceutical composition according to any one of claims 13 to 17 comprising gabapentin and carprofen.

22. A pharmaceutical composition according to any one of claims 13 to 17 comprising pregabalin and carprofen.
